(19) United States
(12) Patent Application Publication
(10) Pub. No.: US 2005/0288247 A1
(43) Pub. Date: Dec. 29, 2005

(54) Inducible inactivation of synaptic transmission

(75) Inventors: Karel Svoboda, Huntington, NY (US); Alla Karpova, Huntington, NY (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: Cold Spring Harbor Laboratory

(21) Appl. No.: 11/166,941

(22) Filed: Jun. 24, 2005

Related U.S. Application Data
(60) Provisional application No. 60/582,995, filed on Jun. 25, 2004.

(51) Int. Cl. A61K 48/00; C12N 5/08; C12N 15/85

(52) U.S. Cl. 514/44; 435/455; 435/368

(57) ABSTRACT

The invention provides molecular systems for inducible and reversible inactivation of synaptic transmission. These systems can be used for studying neuronal networks and for treating conditions involving abnormally high neuronal activity or excitotoxic damage.

This application claims priority from U.S. Provisional Application 60/582,995, filed Jun. 25, 2004, which is incorporated herein by reference in its entirety.

The invention was funded in part by Grant No. 5R01MH070052-03 from the National Institutes of Health. The government may have certain rights in this invention.

### FIELD OF THE INVENTION

This invention relates to neurobiology. More particularly, it relates to molecular systems for inducible and reversible inactivation of synaptic transmission and methods for their use, including treating diseases involving abnormally high neuronal activity or excitotoxic damage.

### BACKGROUND OF THE INVENTION

Complex neuronal circuits consisting of diverse cell types underlay the function of the brain. Relating the activity in specific neuronal circuits to physiological phenomena, behavior, and disease states requires tools that allow modulation of the function of particular types of neurons. Therefore, there has been significant interest in developing approaches for inactivating neuronal activity in a cell-type specific manner (Marek et al., Curr. Opin. Neurobiol. 13:607-11 (2003); Miesenbock, Curr. Opin. Neurobiol. 14:395-402 (2004)).

One approach that has been used is to produce localized lesions by using, e.g., an electrode or a laser. This approach has major drawbacks: the lesions are not reversible, do not select for cell types with adequate specificity, can perturb projections that span the lesion, and are associated with massive compensatory changes.

Other approaches interfere with neuronal excitation or synaptic transmission. The simplest of these approaches involves expression of K⁺ or Cl⁻ channels in a subpopulation of neurons. However, the results can be unpredictable. In the mammalian brain, expression of potassium channels can cause hyperexcitability, rather than hypoexcitability (Sutherland et al., Proc. Natl. Acad. Sci. U.S.A. 96:2451-2455 (1999)). In other cases, silenced neurons undergo apoptosis (Nadeau et al., J. Neurophysiol. 84:1062-1075 (2000)). To introduce temporal control, K⁺ or Cl⁻ channels have been coupled to ligands without endogenous receptors, but these methods have so far only been demonstrated in vitro (Cully et al., Nature 371:707-11 (1994); Lechner et al., J. Neurosci. 22:5287-90 (2002); Li et al., FEBS Lett. 528:77-82 (2002); Scearce-Levie et al., Trends Pharmacol. Sci. 22:414-20 (2001); Slimko et al., J. Neurosci. Methods 124:75-81 (2003); Slimko et al., J. Neurosci. 22:7373-79 (2002)). Further, these methods lead to irreversible or slowly reversible suppression of synaptic transmission as well as non-specific effects, for example, by overloading the energy metabolism (Attwell and Laughlin, J. Cereb. Blood Flow Metab. 21:c 133-45 (2001)).

Expression of a temperature-sensitive mutation of shibire (dynamin) in Drosophila allows conditional silencing of synaptic transmission (Kawasaki et al., Nat. Neurosci. 3: 859-60 (2000)). This has become a useful tool for studying neurotransmitter secretion and the neurobiological basis of behavioral plasticity (Dubnau et al., Nature 411:476-80 (2001); Kawasaki et al., Nat. Neurosci. 3:859-60 (2000); Kitamoto, J. Neurobiol. 47:81-92 (2001); Kitamoto, Proc. Natl. Acad. Sci. U.S.A. 99:13232-37 (2002); McGuire et al., Science 293:1330-33 (2001)). However, this technique is not readily transferable to mammals, where specific inactivation of synaptic activity has been limited to transcriptionally inducible expression of a transgene. For example, modulation of synaptic activity was achieved by expressing a transgene encoding an activated calcium-independent form of calcium-calmodulin-dependent kinase II (Mayford et al., Science 274:1678-83 (1996)) or a tetanus toxin light chain (Yu et al., Neuron 42:553-66 (2004); Yamamoto et al., J. Neurosci. 23:6759-67 (2003)). But those approaches suffer from slow induction and reversal (days to weeks).

### SUMMARY OF THE INVENTION

The present invention is based on the surprising discovery that chemically induced oligomerization of certain neuronal proteins reversibly inactivate synaptic transmission. We have called the systems that we have developed to accomplish this “molecular systems for inactivation of synaptic transmission” or “MISTs.” MISTs allow rapidly inducible inactivation of synaptic transmission in vitro and in vivo (e.g., in a mammal such as a human, a mouse, or a rat). The inactivation is reversible. MISTs are useful for a variety of purposes, including study of neuronal networks and the treatment of diseases that involve abnormally high neuronal activity or excitotoxic damage.

Accordingly, the invention provides a method for inhibiting synaptic transmission of a neuron. In this method, one introduces into a neuron a polynucleotide encoding a fusion protein comprising (i) a ligand-binding domain that binds to a selected ligand, and (ii) a synaptic protein domain, wherein the selected ligand binds to and oligomerizes the fusion protein; and administers to the neuron the selected ligand to oligomerize the fusion protein, thereby inhibiting synaptic transmission of the neuron. The synaptic protein domain comprises a synaptic protein (or a functional portion thereof), i.e., a protein involves in synaptic transmission, especially a protein involved in exocytosis and cycling of synaptic vesicles. Examples of synaptic proteins include, without limitation, Synaptobrevin/VAMP2, SNAP-25, Syntaxin, Synaptophysin, or RIM1. Examples of ligand-binding domains include FK506-binding protein (FKBP) or variants thereof.

The invention provides another method for inhibiting synaptic transmission of a neuron. In this method, one introduces into a neuron (1) a first polynucleotide encoding a first fusion protein comprising (i) a first ligand-binding domain that binds to a selected ligand, and (ii) a synaptic protein domain, and (2) a second polynucleotide encoding a second fusion protein comprising (i) a second ligand-binding domain that binds to the selected ligand, and (ii) a mislocalizer domain, wherein the selected ligand binds to and crosslinks the first and second fusion proteins. One can then administer to the neuron the selected ligand to crosslink the first and second fusion proteins, thereby inhibiting synaptic transmission of the neuron. The synaptic protein domain may comprise a synaptic vesicle protein such as Synaptophysin or Synaptobrevin/VAMP2. The mislocalizer domain comprises a protein (or a functional portion thereof) that, when crosslinked to the first fusion protein, sequesters the first fusion protein in a place that prevents the normal fusion between synaptic vesicles and the plasma membrane. Examples of mislocalizer domains include those comprising a cell surface protein (or a transmembrane domain thereof) such as Syntaxin or a cytoskeletal matrix protein (or a functional portion thereof) such as RIM1. The first and second ligand-binding domains are FKBP and the FKBP-binding domain of FRAP (FRB), respectively, or FRB and FKBP, respectively (or variants thereof).

In some embodiments, the inactivation methods of this invention can be used to treat a disease, disorder or condition in a mammal, wherein the disease, disorder or condition involves abnormally high neuronal activity or excitotoxic damage.

The invention further provides a method for identifying a synaptic protein whose oligomerization inhibits synaptic transmission of a neuron. This method comprises: providing a neuron comprising a polynucleotide encoding a fusion protein comprising (i) a ligand-binding domain that binds to a selected ligand, and (ii) a domain comprising a candidate synaptic protein, wherein the selected ligand binds to and oligomerizes the fusion protein; administering to the neuron the selected ligand to oligomerize the fusion protein; and detecting synaptic transmission of the neuron, wherein reduction in the transmission indicates that the candidate synaptic protein inhibits synaptic transmission of the neuron when oligomerized.

This invention also provides a method of identifying a synaptic protein whose crosslinking to another cellular protein inhibits synaptic transmission of a neuron, the method comprising: providing a neuron comprising (1) a first polynucleotide encoding a first fusion protein comprising (i) a first ligand-binding domain that binds to a selected ligand, and (ii) a domain comprising a candidate synaptic protein (or a functional portion thereof), and (2) a second polynucleotide encoding a second fusion protein comprising (i) a second ligand-binding domain that binds to the selected ligand, and (ii) a domain comprising the cellular protein (or a functional portion thereof), wherein the selected ligand binds to and crosslinks the first and second fusion proteins; administering to the neuron the selected ligand to crosslink the first and second fusion proteins; and detecting synaptic transmission of the neuron, wherein reduction in the transmission indicates that the candidate synaptic protein inhibits synaptic transmission of the neuron when crosslinked to the cellular protein.

### BRIEF DESCRIPTION OF THE DRAWINGS

FIG. 1 is a schematic of exemplary fusion proteins used herein. These include a one-component MIST (based on homodimerization of Synaptobrevin/VAMP2 “VAMP/Syb”) and a two-component MIST (based on heterodimerization of Synaptophysin and the transmembrane domain of Syntaxin). Abbreviations are as follows. SV: synaptic vesicle; PM: plasma membrane of the neuron; VAMP: VAMP/Syb; Sph: Synaptophysin; Stx-TM: transmembrane domain of Syntaxin 1A; FKBP: FK506-binding protein; FKBP*: FKBP with a Phe36Val mutation; FRB: FKBP-binding domain of FRAP.

### DETAILED DESCRIPTION OF THE INVENTION

We have discovered methods for inducibly and reversibly inactivating synaptic transmission in genetically targeted neuronal populations. These methods are based on proteins involved in synaptic transmission (i.e., synaptic proteins), especially those involved in exocytosis and cycling of synaptic vesicles. Synaptic vesicles are generated inside a neuron and contain neurotransmitters. When the vesicles fuse with the plasma membrane of the neuron in a so-called “active zone,” the neurotransmitters are released to the synaptic cleft between the neuron (presynaptic neuron) and its post-synaptic neighbor (Sudhof, Annu. Rev. Neurosci. 27:509-47 (2004)). Fusion of the vesicle membrane and the plasma membrane is mediated by the SNARE complex. The complex contains at least one protein from the synaptic vesicle—Synaptobrevin, also called vesicle-associated membrane protein (VAMP)— and at least two proteins from the plasma membrane—Syntaxin 1 and SNAP-25. SNARE complex formation and vesicle formation is controlled by a number of proteins, including Synaptophysins, Sec1/Munc18-like proteins, tomosyn, amisyn, complexins, and RIM1.

We have discovered that engineered synaptic proteins containing an oligomerizer-binding domain (e.g., a FKBP, which binds to oligomerizer FK506 or a derivative thereof) perturbs, upon oligomerization in a neuron, the exocytosis and cycling of synaptic vesicles in that neuron through a heretofore unknown mechanism. As a result, synaptic transmission from that neuron is blocked. The blockage is reversed when the oligomerizer (i.e., oligomerization inducer) is removed. We have discovered that when the engineered synaptic proteins are expressed in Purkinje cells in a mammal, the proteins' oligomerization inducibly and reversibly alters the mammal's behaviors that require normal cerebellar function. Without being bound by any theory, we hypothesize that in at least some cases, the oligomerized engineered synaptic proteins have a dominant negative effect on their corresponding endogenous counterparts. In our invention, useful synaptic proteins include synaptic vesicle proteins (i.e., proteins that are in or on synaptic vesicles) such as VAMP/Syb, Synaptophysins, Synaptogyrins, SV2 proteins, and Synaptotagmins; plasma membrane proteins involved in vesicle fusion such as Syntaxins; and components of the cytoskeletal matrix in the active zone such as RIM1, Bassoon, Piccolo, and Muncl3.

We have discovered that blockage of synaptic transmission can also be achieved by crosslinking between an engineered synaptic protein and a cellular component such as a plasma membrane protein and a cytoskeletal matrix protein. The crosslinking takes place when the engineered synaptic protein binds via its crosslinker-binding domain (e.g., a FKBP, which binds to crosslinker or hetero-oligomerizer AP21967 (ARIAD)) to an engineered cellular component which also has a domain that binds to the crosslinker (e.g., a FRB, which also binds AP21967). The blockage is reversed when the crosslinker is removed. Without being bound by any theory, we hypothesize that at least in some of these cases, crosslinking sequesters synaptic vesicles or elements involved in synaptic vesicle exocytosis and cycling in inappropriate places in the neuron, thereby preventing fusion between synaptic vesicles (SV) and the plasma membrane. The fusion between SV and the plasma membrane takes place in a very confined area in a neuron. Thus even a minor perturbation in the movement of SV may interference with their fusion with the plasma membrane. We call the engineered cellular component a “mislocalizer” for sequestering synaptic vesicles or elements at an inappropriate cellular location. Mislocalizers useful in this invention include cell surface proteins such as Syntaxin or its transmembrane domain (e.g., amino acids 259-288 of rat Syntaxin 1A) and cytoskeletal matrix proteins such as RIM41, Munc13 and actin.

Our invention provides an ideal tool for inducible and reversible perturbation of neural activity. The inactivating effect of the engineered proteins only exists when the corresponding oligomerizer is introduced into the neuron, and that effect disappears when the oligomerizer is removed or metabolized. Moreover, MISTs work qualitatively differently than other lesion and inactivation technologies, such as electrolytic lesions, or pharmacological manipulations, such as muscimol or lidocaine. The onset of the inactivation of synaptic transmission is rapid—it is typically less than ten minutes on average in dissociated neurons and acute brain slices and hours in vivo, compared to days or weeks in the conventional inactivation methods. The reversal process can also occur rapidly. For example, the VAMP/Syb MIST described in detail below allows full recovery of synaptic transmission in dissociated cultures within 12 hours and of test animals' behavioral task performance within 36 hours. This recovery time course can be further sped up by the delivery of a specific antagonist.

MISTs have many applications, e.g., in studies of the role of particular classes of neurons in vivo and in vitro, including animal behavioral study; in investigations of the presynaptic vesicle cycle; in treatment of neurological disorders of neuronal hyperactivity and as a tool for neuroengineering.

Transgenic approaches to gene delivery of MIST constructs will allow functional studies of specific genetically-defined classes of neurons (different interneurons, neuromodulatory systems, etc.). Use of two-component MISTs driven by different promoters will lead to higher spatial specificity of the effect since inactivation occurs only in the intersection of the two expression patterns. Also, localized infections by viral-based MIST constructs permits analysis of specific brain regions.

Another unique opportunity offered by the instant invention is in understanding the role of activity in various circuits during embryonic development. Lesions or localized injections of muscimol or lidocaine are impossible in utero. Appropriate transgenic targeting of MIST expression combined with subcutaneous delivery of the oligomerizers to the mother will allow control over embryonic neurotransmission as oligomerizers can cross the placenta.

Further, MISTs can be used to selectively silence specific neuronal projections. Many neurons in the central nervous system project to multiple target areas. It is critical to be able to separate the contributions of the different projections. For example, it has been difficult to assess the role of corticothalamic feedback on cortical processing. If a MIST is expressed in cortical pyramidal neurons, localized injection of the corresponding oligomerizer at the terminals of a particular projection, in this case in thalamus, will allow selective silencing of corticothalamic feedback.

In addition, MISTs provide a basis for the dissection of the synaptic vesicle cycle and for the specific manipulation of particular presynaptic players that has distinct advantages over previous approaches. For example, genetic approaches, such as knock-outs, can be inducible and specific but are irreversible and slow. Toxins, such as botulinum toxin, and fluorescent light fluorophore assisted light inactivation (FALI) permit inducible, specific and rapid manipulations of presynaptic targets, but the effects are irreversible. Further, only particular presynaptic proteins are targets of toxins. Using a library of putative MISTs based on presynaptic proteins it will be possible to discover other players whose perturbation will lead to the inactivation of neurotransmission. In addition, other properties of neurotransmission may be inducibly and reversibly affected by the different MISTs, such as short-term plasticity, long-term potentiation and depression, synaptogenesis and synaptic disassembly. MISTs therefore provide a set of tools for the exploration of the molecular basis of presynaptic function.

MISTs can be further used for gene therapy in neurological disorders characterized by abnormally high neuronal activity or excitotoxic damage, including epilepsy, Parkinson's disease, Huntington's disease, amyotrophic lateral sclerosis, dystonia, stroke, myoclonus, tic disorders, chorea, spasticity, tremor, pain, hallucination, dyskinesia, chronic anxiety, obsessive-compulsive disorder, and schizophrenia. In these disorders, drugs that reduce synaptic activity, including benzodiazepines, calcium and sodium channel blockers, or drugs that reduce glutamate excitotoxicity have been shown to significantly ameliorate symptoms and possibly provide neuroprotection (Doble, Pharmacol. Ther. 81:163-221 (1999) and references cited therein). Viral delivery of a genetically-encoded silencing system to affected parts of the nervous system allows more specific, tunable, and reversible treatment than current approaches.

Neuroengineering may also benefit from the use of MISTs. For example, neurons and neuronal networks have been shown to be useful in a number of different areas of engineering such as biosensing and computation. MISTs allow the dynamic control of such devices by allowing the inducible and reversible control of input, output and computations.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. The materials, methods, and examples are illustrative only and not intended to be limiting. In order to further define this invention, the following terms and definitions are provided.

As used herein, a “mammal” includes, e.g., a primate (e.g., a human, a monkey, or a chimpanzee), a rodent (e.g., a rat, a mouse), a rabbit, or a guinea pig.

As used herein, a “fusion protein” means a protein comprising a first polypeptide fused to a second, heterologous, polypeptide.

As used herein, an “oligomerizer” is a chemical compound that has more than one (e.g., 2, 3, 4, or 5) high affinity binding sites for one or more binding polypeptides.

As used herein, a “homodimerizer” is a dimerizer that has two high affinity binding sites for a dimerizer-binding polypeptide and, accordingly, a homodimerizer induces dimerization of two separate molecules of the same dimerizer-binding polypeptide.

As used herein, a “heterodimerizer” is a dimerizer that has two high affinity binding sites for two distinct dimerizer-binding polypeptides and, accordingly, a heterodimerizer induces dimerization of two different dimerizer-binding polypeptides.

As used herein, a “reverser” is a chemical compound that binds to the same site on a dimerizer-binding polypeptide as a cognate dimerizer and, accordingly, competes with the dimerizer for binding to the dimerizer-binding polypeptide.

Oligomerizable Synaptic Fusion Proteins

The oligomerizable synaptic fusion proteins of this invention comprise a domain that binds to a selected oligomerizer (e.g., a dimerizer), and a domain comprising a synaptic protein (or a portion thereof). A large number of oligomerizer-binding polypeptides and their cognate oligomerizers are known in the art and useful in this invention. See, e.g., Crabtree and Schreiber, Trends. Biochem. Sci. 21:418-22 (1996); Rivera, Methods 14:421-29 (1998); Spencer et al., Science 262:1019-24 (1993); and Crabtree U.S. Pat. No. 5,830,462. These systems have been used in mammals both in vitro and in vivo. See, e.g., Volchuk et al., Cell 102:335-48 (2000); Rivera et al., Science 287:826-30 (2000); Moskowitz et al., Mol. Biol. Cell. 14:4437-47 (2003); Muthuswamy et al., Nat. Cell. Biol. 3:785-92 (2001); Jin et al., Nat. Genet. 26:64-66 (2000); Mallet et al., Nat. Biotechnol. 20:1234-39 (2002). The oligomerizers used in this invention are typically organic molecules that are freely able to cross the plasma membrane of intact cells. Generally, oligomerizers that do not interact with any endogenous proteins are used so that cytotoxicity can be avoided.

In a one-component MIST, the fusion protein may comprise a synaptic protein fused to a heterologous polypeptide that is able to bind specifically to a homodimerizer. In a two-component MIST, a synaptic protein and a mislocalizer (which can be a synaptic protein itself) may be separately fused to two different heterologous polypeptides, each of the heterologous polypeptides being able to bind specifically to the same heterodimerizer. The synaptic protein domain in the fusion protein need not comprise the full-length sequence of the cognate synaptic protein. Generally, however, a sufficient portion of the cognate synaptic protein is present so that the fusion protein retains enough of the desired wild-type activity.

Fusion proteins of this invention can be constructed in several different configurations. In one configuration, the C-terminus of the synaptic protein is fused directly to the N-terminus of the dimerizer-binding polypeptide. In a slightly different configuration, a short spacer polypeptide, e.g., 2-10 amino acids, is incorporated into the fusion between the C-terminus of the neuronal protein and the N-terminus of the dimerizer-binding polypeptide. Such a spacer provides conformational flexibility, which may improve biological activity in some circumstances. The fusion proteins may also comprise detectable moieties such as a myc tag and a fluorescent or luminescent protein domain, and/or moieties, such as a signal peptide or a myrisylation site, that direct the proteins to the desired cellular location.

Vectors

The choice of vector for making the MIST constructs and expression control sequences to which the fusion protein-encoding sequences are operably linked depends on the functional properties desired and the neuronal cell target to be transformed.

The expression control elements in the MIST constructs generally allow high-level expression of the fusion protein. Accordingly, the fusion protein is present in much greater numbers in the target neurons than the corresponding endogenous synaptic protein. Many expression control elements useful for regulating the expression of operably linked coding sequences are known in the art. Frequently used regulatory sequences for mammalian expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus (e.g., the adenovirus major late promoter (AdMLP)), polyoma and strong mammalian promoters such as native immunoglobulin and actin promoters. For further description of viral regulatory elements and sequences thereof, see, e.g., Stinski, U.S. Pat. No. 5,168,062; Bell, U.S. Pat. No. 4,510,245; and Schaffner, U.S. Pat. No. 4,968,615. Generally, expression control elements that are constitutively expressed are used. However, inducible expression control elements and other regulatory elements may be used if their expression level is sufficient so that the fusion protein predominates in the cell. When an inducible promoter is used, it can be controlled, e.g., by a change in nutrient status. In some embodiments, promoters that target expression to specific neuronal cell types or populations are used.

In some embodiments, the fusion protein is produced in vivo in a mammal, e.g., a human patient, using a gene-therapy approach. This generally involves the use of viral based vector. Suitable viral vectors include adenoviral vectors, lentiviral vectors, Epstein Barr viral vectors, papovaviral vectors, vaccinia viral vectors, herpes simplex viral vectors, and adeno-associated virus (AAV) vectors. The viral vector can be a replication-defective viral vector. Adenoviral vectors that have a deletion in its E1 gene or E3 gene can be used.

Gene Therapy

In some embodiments, the fusion protein is produced in vivo in a mammal to treat diseases, disorders or conditions where the principal symptoms result from over-activity of a population of neurons, e.g., epilepsy due to excessive firing of neurons in the cerebral cortex. In addition, the methods may be used to treat diseases, disorders or conditions in which silencing a population of neurons is clinically useful, e.g., Parkinson's disease, where suppressing the activity of neurons in the subthalamic nucleus (STN) relieves dopaminergic-therapy-induced dyskinesia. Diseases, disorders or conditions that can be treated using the methods of the invention include, but are not limited to, epilepsy (including primary syndromes of generalized and partial seizures as well as secondary epilepsies), dystonia (including primary dystonias for which botulinum toxin injections and benzodiazepines are helpful (such as DYT-1 dystonia) as well as some secondary dystonias (e.g., due to cerebral palsy)), myoclonus, tic disorders (including Gilles de la Tourette syndrome), chorea (including Huntington's disease), spasticity (for example in multiple sclerosis or after stroke or spinal cord injury), tremor, pain, hallucinations (for example in dementia or Parkinson's disease), and tardive dyskinesia (including other tardive disorders). In addition, the methods of the invention can be used to treat symptoms that are currently treated with benzodiapines, including chronic anxiety, obsessive-compulsive disorder, and schizophrenia.

The oligomerizers used in the methods of the invention may be formulated into pharmaceutical compositions for administration to patients. The pharmaceutical compositions used in the methods of this invention may comprise pharmaceutically acceptable carriers known in the art. The compositions used in the methods of the present invention may be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

When the brain is the target for the therapy, the oligomerizers are chosen or administered in such a way that they cross the blood-brain barrier. This crossing can result from physico-chemical properties inherent in a particular oligomerizer, from other components in a pharmaceutical formulation, or from the use of a mechanical device such as a needle, cannula or surgical instruments to breach the blood-brain barrier by intrathecal or intracranial administration. For example, this can be done with a stereotactically implanted pump, temporary interstitial catheters, permanent intracranial catheter implants, and surgically implanted biodegradable implants. See, e.g., Gill et al., Nature Med. 9:589-95 (2003); Scharfen et al., Int. J. Radiation Oncology Biol. Phys. 24(4):583-91 (1992); Gaspar et al., Int. J. Radiation Oncology Biol. Phys. 43(5):977-82 (1999); Bellezza et al., “Stereotactic Interstitial Brachytherapy,” in Gildenberg et al., Textbook of Stereotactic and Functional Neurosurgery, Chapter 66, pages 577-580 (McGraw-Hill 1998); and Brem et al., J. Neuro-Oncology 26:111-23 (1995).

The amount of an oligomerizer that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response). The treatment methods of the invention use a “therapeutically effective amount” of an oligomerizer. Such a therapeutically effective amount may vary according to factors such as the extent of production of the fusion protein(s) in the target cells and the disease state, age, sex, and weight of the individual. As described above, oligomerizers are generally non-toxic or minimally toxic. However, a therapeutically effective amount is also one in which any toxic or detrimental effects are outweighed by the therapeutically beneficial effects. Generally, compositions for administration according to the methods of the invention can be formulated at a dosage of 0.001-10 mg/kg body weight per day. In some embodiments, the dosage is 0.01-1.0 mg/kg body weight per day. In some embodiments, the dosage is 0.001-0.5 mg/kg body weight per day.

### EXAMPLES

The following examples are meant to illustrate the methods and materials of the present invention. Suitable modifications and adaptations of the described conditions and parameters normally encountered in the art are within the spirit and scope of the present invention.

### Example 1

### Generation of Dimerizable Fusion Proteins of Neuronal Proteins

Results

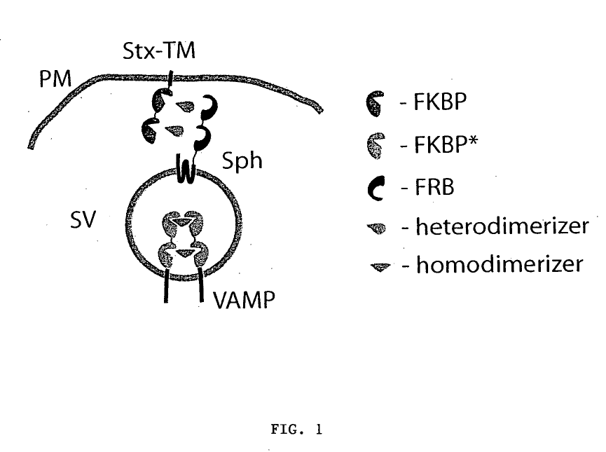

Many proteins in the synaptic terminal are both essential and specific for the process of synaptic transmission (Femandez-Chacon and Sudhof, Ann. Rev. Physiol. 61: 753-776 (1999); Sudhof, Ann. Rev. Neurosci. 27:509-47 (2004)). Given the complex spatial-temporal sequence of protein-protein interactions leading to synaptic vesicle fusion, we reasoned that sequestration of an essential player by chemical-induced dimerization (CID) might cause perturbation of synaptic transmission. Potential MISTs fall into at least two classes: those that rely on inducible crosslinking of synaptic vesicle (SV) proteins and those that rely on sequestering synaptic proteins away from their site of action. Based on genetic and biochemical data alone it was difficult to predict an effective strategy for silencing of synaptic transmission by CID. We therefore generated and tested a panel of about 30 candidate MISTs. Examples are illustrated in FIG. 1.

These MISTs were based on oligomerization of synaptic proteins resulting in (1) intra-vesicular crosslinking (e.g., homodimerization of VAMP/Syb), (2) inter-vesicular crosslinking (e.g., homodimerization of Synaptophysin (“Sph”), (3) crosslinking of synaptic vesicles to mitochondria (e.g., heterodimerization of Sph and Bcl-2 (“Sph-Bcl2”), (4) crosslinking of synaptic vesicles to the plasma membrane (e.g., heterodimerization of Sph to the syntaxin transmembrane domain (“Sph-StxTM”), (5) crosslinking of the active zone cytoskeletal protein RIM1 (“RIM”), or (6) crosslinking of synaptic vesicles to the active zone (e.g., heterodimerization of Sph and RIM1 (“Sph-RIM”).

Specifically, we created the following one-component MIST fusion constructs: VAMP-FKBP*2 (2: two FKBP* domains), SNAP25PD-FKBP*2 (PD: palmitoylation domain) and FKBP2-Sph-GFP-FRB2 (GFP: green fluorescent protein). We created a number of two-component MISTs, using each one of Sph-GFP-FRB2, Munc18-GFP-FRB2, Munc13-GFP-FRB2, and myc-FRB2-VAMP (myc: myc epitope tag), with each one of the following mislocalizer partners: myc-FKBP2-StxTM, SNAP25PD-FKBP2, myc-FKBP2-bcl-2, myc-FKBP2-actin, and myr-FKBP3 (myr: myrisylated).

In the above two-component MIST systems, Stx-TM, RIM and Bcl-2 were used as mislocalizers to sequestering the synaptic vesicles away from their normal docking sites, therefore interfering with exocytosis. Two-component MISTs provide several advantages. For example, the use of two separate promoters that direct expression only in particular populations of neurons can enhance the specificity of targeting because inactivation will occur only in those cells where the expression of both fusion proteins of the MIST overlaps.

The MISTs in this Example were generated using variants of the dimerizer-binding polypeptides FKBP (which binds a non-toxic derivative of FK506) and/or FRB (which binds rapamycin). We next screened for their ability to inactivate synaptic transmission upon CID.

Experimental Procedures

All FKBP and FRB vectors were obtained from ARIAD Pharmaceuticals (Cambridge, Mass., http://www.ariad.com/regulationkits/index.html). VAMP/Syb MIST was created by fusing rat VAMP/Syb with two domains of FKBP(36V) tagged with hemagglutinin tag (HA). We created an adenoviral vector that expressed VAMP-FKBP(36V)₂-HA-IRES-VenusYFP under the control of the pCAG promoter. Woodchuck hepatitis virus posttranscriptional regulatory element, WPRE, was included in the transgene to enhance expression levels (Zufferey et al., J. Virol. 73:2886-92 (1999); from Dr. T. J. Hope). Adenovirus was produced by ViraQuest Incorporated (North Liberty, Iowa). Viral titer was about 10¹⁰ pfu/ml. We also created a lentiviral vector that expressed VAMP/Syb MIST and EGFP from a bidirectional ubiquitin-based promoter (Amendola et al., 2005).

Synaptophysin-mEGFP-FRB was created by fusing two domains of FRB to the C-terminus of Synaptophysin-EGFP (from Dr. Jane Sullivan), and by substituting the monomeric version of enhanced green fluorescent protein (“EGFP”) (Zacharias et al., Science 296:913-16 (2002)) for the wild type protein in the original fusion. StxTM-based mislocalizer consisted of transmembrane domain of rat Syntaxin 1 (“StxTM”) fused at the C-terminus to myc-tagged FKBP2. We created a plasmid that expressed myc-FKBP2-StxTM-IRES-Sph-mGFP-FRB2. Other MIST constructs were made similarly.

### Example 2

### Dimerization of Fusion Proteins Inhibits Synaptic Transmission in Dissociated Cultured Neurons

Results

We screened for CID-mediated inactivation of synaptic transmission in dissociated cultured neurons. We transfected cells with constructs for candidate MISTs and assayed activity-dependent synaptic vesicle cycling by imaging FM 4-64 fluorescence in synaptic terminals. Within each experiment we established the dynamic range of the assay by measuring FM 4-64 loading with intact vesicle cycling (vehicle; negative control) and vesicle cycling blocked (Cd²⁺; positive control). We further measured FM 4-64 fluorescence in the presence of the dimerizer. We calculated a “block index” (BI, range 0, 1) that measured how close the dimerizer condition was to the positive (BI=1) and negative (BI=0) controls under our stimulus conditions.

The screen revealed two MISTs that significantly reduced synaptic vesicle cycling in the presence of the corresponding dimerizer: (1) homodimerization of VAMP/Syb (BI=0.45±0.05), and (2) heterodimerization of Sph and StxTM (BI=0.59±0.07). For both systems CID-dependent inactivation of synaptic vesicle cycling was reversed after the dimerizer was washed out (Reversal: VAMP/Syb MIST, 97.7%; Sph-StxTM MIST, 50.9%).

We verified (1) by measuring activity-dependent vesicle cycling using FM 4-64 in dissociated cultures transduced with lentiviral vectors expressing VAMP/Syb MIST or EGFP. There were no significant differences in the size of the releasable pools or in the destaining kinetics in the two populations of synapses. Given the ease of transgene delivery and full reversibility of the one-component VAMP/Syb MIST, it was chosen for detailed analysis.

Experimental Procedures

For the FM-dye screen, dissociated hippocampal cultures were prepared from embryonic rat pups (E17-19) as described (Evans et al., J. Neurosci. Methods 79:37-46 (1998)). For FM-dye destaining kinetics measurements, dissociated hippocampal co-cultures were prepared from postnatal rat pups (P2-3) as described (Deak et al., Nat. Cell Biol. 6:1102-08 (2004)). Dissociated cultures were transfected using EFFECTENE (Qiagen, Valencia, Calif.) for the FM-dye screen or infected with lentivirus for the measurement of destaining kinetics at six days in vitro and used for experiments at fourteen days in vitro.

The FM-dye screen consisted of a series of fluid exchanges in six-well plates containing transfected or infected dissociated neurons: preincubation in Neurobasal Medium (Invitrogen, Carlsbad, Calif.), supplemented with 100 μM Cd²⁺, vehicle or dimerizer; stimulation with hyperkalemic solution (30 mM KCl, 2 mM Ca²⁺, 2 mM Mg²⁺ in Tyrodes solution) for 2 minutes; a two-minute rest period; two-minute staining in 30 mM KCl with 10 μM FM 4-64; fifteen minutes wash period using Neurobasal Medium containing CellTracker Blue (Molecular Probes, Eugene, Oreg.) to stain living neurons and then incubated at 37° C. for 20 minutes. Coverslips were then fixed with 4% paraformaldehyde. All solutions contained 10 μM NBQX and 50 μM DL-APV to prevent network activity. All solutions in the positive control condition contained 0.01% (vol/vol) ethanol for homodimerization systems, or 0.001% (vol/vol) DMSO for heterodimerization systems. All solutions in the negative control condition contained 100 μM Cd²⁺ to block synaptic transmission. All solutions in the dimerizer condition contained 10 nM AP20187 for homodimerization experiments, or 100 nM AP21967 for heterodimerization experiments. For reversal experiments, this procedure was performed omitting the FM-dye and then repeated after twelve hours in the absence of vehicle or dimerizer in the presence of FM-dye. Coverslips were imaged immediately after fixation on an Olympus microscope (Olympus, Melville, N.Y.) using a 60× objective (Olympus; 60×; NA 1.3), a Xenon lamp, a CCD (Orca ER II; Hamamatsu, Hamamatsu City, Japan), and commercial software (Slidebook; Intelligent Imaging Innovations, Inc., Denver, Colo.).

Each FM-dye screen experiment consisted of three to six coverslips of hippocampal dissociated neurons in the vehicle and drug conditions and two coverslips in the Cd²⁺ condition. Coverslips were analyzed blind to whether dimerizer or vehicle had been added in the experiment. Up to ten fields of view, measuring 8991 μm by 6605 μm each, were selected randomly from each coverslip. In the vehicle and drug conditions, at least 150 axon segments were selected for analysis from these fields of view, again blind to the condition. All axon segments were selected from each field of view, unless they obviously overlapped with non-specific fluorescent background. The peaks on these axon segments were then detected, yielding 1000-1500 peaks both for the vehicle and drug conditions for each experiment. These peaks were then averaged in each condition, and used to calculate the blocking index.

To quantify the inhibition of FM-dye staining in the dimerizer condition, we used custom software programmed in Matlab (Mathworks, Natick, Mass.) to detect and measure the FM 4-64 staining on transfected axons. The mean of the FM-dye peaks in each condition were used to estimate extent of block:

$$Block index = \frac{〈 {FM}_{vehicle} 〉 - 〈 {FM}_{drug} 〉}{〈 {FM}_{vehicle} 〉 - 〈 {FM}_{{Cd}^{2 +}} 〉}$$

The size of the readily releasable pool and FM-dye destaining kinetics were measured using field stimulation and timelapse imaging (Murthy et al., Neuron 18:599-612 (1997); Ryan and Smith, Neuron 14:983-89 (1995)). Dissociated hippocampal co-cultures infected with lentivirus carrying either EGFP or VAMP/Syb-MIST were exposed to 10 μM FM 4-64, stimulated at 30 Hz for 1 min using field stimulation (S48 Stimulator, Grass Medical Instruments, Quincy, Mass.; 1 msec duration at 20 V/cm across platinum electrodes), exposed to FM 4-64 for an addition 30 seconds and than washed for 15 minutes in Tyrodes solution. All solutions contained 10 μM NBQX and 10 μM CPP to block recurrent activity. Fields of view were then randomly selected, and destained at 30 Hz stimulation, while acquiring images at 0.2 Hz. Quantitative measurements of the destaining kinetics were obtained by manually selecting ROIs of 3×3 pixels in area (approximately 0.7 μm×0.7 μm). The pixel intensity in each ROI was averaged, and a fluorescence change for each ROI was calculated by subtracting the baseline intensity from the mean intensity of the final three images. The rate of perfusion was approximately 1 ml/min and all experiments were done at room temperature.

The above FM dye experiment can also be carried out in the conventional longitudinal way, where the same neurons are observed before and after the dimerizer treatment.

### Example 3

### Dimerization of Fusion Proteins Inhibits Synaptic Transmission in Brain Slices

Results

We first tested the VAMP MIST construct in cultured brain slices. The adenovirus-based VAMP MIST construct was used to infect cortical neurons in cultured brain slices. Synaptic transmission was evoked with extracellular stimulation. The resulting excitatory postsynaptic currents (“EPSCs”) often had a monosynaptic component followed by a network discharge. Addition of homodimerizer (20 nM AP20187 in DMSO; ARIAD Pharmaceuticals, Cambridge, Mass.) to the bath caused a rapid decrease in the amplitudes of both the monosynaptic EPSC and the network discharge. Addition of a reverser (200 nM AP21998; ARIAD Pharmaceuticals, Cambridge, Mass.) caused rapid recovery of both the monosynaptic EPSC and the network discharge.

We further characterized CID-dependent inactivation of synaptic transmission using whole cell measurements of EPSC in layer (L) 2 pyramidal neurons in acute brain slices. MIST constructs were introduced into L 3 cells in vivo using in utero viral vector mediated gene delivery (VAMP/Syb MIST) or electroporation (Sph-StxTM MIST). Synaptic transmission was evoked with an extracellular stimulation electrode placed in a band of transfected/infected neurons, below (within 200 μm) the recording pipette. Bath application of either heterodimerizer for the Sph-StxTM MIST (500 nM AP21967), or homodimerizer for the VAMP/Syb MIST (100 nM AP20187) reliably caused a rapid decrease in the amplitude of monosynaptic EPSCs. Addition of an excess of monomeric reverser (5 μM AP21998) caused rapid partial recovery of the response. While inactivation of synaptic transmission was reliably observed (range 50-100%), the time course of inactivation varied significantly (10-30 minutes).

The observed inactivation was specific to treatment with the chemical dimerizers, as addition of the vehicle had no effect on synaptic transmission. The inactivation was also specific to neurons expressing the MISTs since even at much higher doses (1 μM) the dimerizers had no effect on synaptic transmission in uninfected slices. In a subset of experiments we applied an excess of reverser (5 μM AP21998), which resulted in partial recovery of synaptic transmission in minutes.

Experimental Procedures

Cortical brain slices (300 μm thick) were prepared from young mice (postnatal day 9-14) using a vibrating-blade microtome (VT1000S, Leica Microsystems). Chilled cutting solution contained 110 mM choline chloride, 25 mM NaHCO₃, 25 mM D-glucose, 11.6 mM sodium ascorbate, 7 mM MgSO₄, 3.1 mM sodium pyruvate, 2.5 mM KCl, 1.25 mM NaH₂PO₄, and 0.5 mM CaCl₂. Slices were then transferred to artificial cerebrospinal fluid (“ACSF”) containing 127 mM NaCl, 25 mM NaHCO₃, 25 mM D-glucose, 2.5 mM KCl, 2 mM MgCl₂,1 mM CaCl₂, and 1.25 mM NaH₂PO₄, aerated with 95% O₂/5% CO₂. Slices in ACSF were first incubated at 34° C. for 0.5 hr, then maintained at room temperature prior to use. For recordings, ACSF contained 4 mM MgCl₂, 4 mM CaCl₂ and 5 M CPP.

Neurons were visualized with infrared differential interference contrast optics, and patched using borosilicate electrodes (resistances, 4-7 MΩ). Access resistances were in the range 10-30 MΩ. Intracellular solution contained 120 mM CsMeSO₃, 20 mM CsCl, 4 mM NaCl, 10 mM HEPES, 10 mM BAPTA, 4 mM Mg₂ATP, and 0.3 mM Na₂GTP, 14 mM sodium phosphocreatine, 3 mM ascorbate, and 0.1 Alexa-594 (Molecular Probes); pH was adjusted to 7.25 with CsOH. Excitatory currents were measured at a holding potential of −65 mV, close to reversal for fast inhibition. MIST-expressing neurons were stimulated with tungsten bipolar electrode in the middle of transfected region, and monosynaptic responses were recorded from cells within 200 μM. Responses were amplified (Multiclamp, Axon Instruments), filtered at 1 kHz, and digitized at 10 kHz. Custom software written in Matlab (MathWorks, Natick, Mass.) was used for electrophysiological acquisition and data analysis.

Homodimerizer AP20187, and heterodimerizer AP21967 were dissolved in DMSO to a final concentration of 100 μM and 500 μM, respectively. Following acquisition of baseline evoked responses, the appropriate dimerizer was added to the perfusion at 1:1000 dilution for final concentrations of 100 nM and 500 nM. The monomeric antagonist, AP21998, was added to 5 μM.

For in utero transgene delivery, in utero surgery was performed as described (Saito and Nakatsuji, Dev. Biol 240:237-46 (2001)). In brief E15.5-E16.5 timed-pregnant mice were kept anaesthetized with continuous flow of isoflurane/oxygen mix, and adenovirus expressing VAMP/Syb MIST or plasmid expressing myc-FKBP2-StxMT-IRES-Sph-mGFP-FRB2 were injected using glass micropipettes into the lateral ventricle of embryos through the uterine wall. For plasmid electroporation, square electric pulse was applied to the embryos by holding them with forceps-type electrodes along the anterior-posterior axis.

### Example 4

### Dimerization of Fusion Proteins Alters Animal Behavior

Results

We further tested the VAMP/Syb MIST in the context of behavioral experiments in vivo. We focused on inactivation of Purkinje cell output. Purkinje cells provide the output from the cerebellar cortex to the deep cerebellar nuclei, a projection that is believed to be involved in motor learning and motor performance (Thach et al., Ann. Rev. Neurosci 15:403-42 (1992)). Ablation of Golgi cells (Watanabe et al., Cell 95:17-27 (1998)) or reduction of synaptic transmission from Granule to Purkinje cells (Hirai et al., Nat. Neurosci 6:869-76 (2003); Yamamoto et al., J. Neurosci. 23:6759-67 (2003)) lead to motor deficits. In addition, mice with Purkinje cell degeneration show reduced performance in a rotarod balance test correlating with the disease progression (Lalonde et al., Neurobiol. Learn. Mem. 65: 113-20 (1996)).

In our experiment, we expressed VAMP/Syb MIST under the control of the L7 promoter in transgenic mice (Zhang et al., Histochem Cell Biol 115:455-64 (2001)). The transgene was specifically expressed in most cerebellar Purkinje cells. Transgenic mice were acquainted with the Rotarod task. And then a cannula was implanted for delivery of the drug into the lateral ventricle. After several days of recovery, Rotarod performance was scored first in baseline conditions, and then following intra-ventricular delivery of either homodimerizer or vehicle.

The data showed that transgene expression by itself (i.e., in the absence of dimerizer) had no effect on the average latency to fall in the transgenic animals. However, significant transient decrease in performance was observed after delivery of drug but not after delivery of the vehicle alone, during the learning phase of the task. One session (24 hours) later learning resumed at almost normal rates. Following saturation of learning, CID caused a transient decrease in Rotarod performance, which was reversed by 36 hours after injection. In either type of experiment, no decrease in performance was observed in wild-type animals. In experiments performed blindly before genotyping, we found that for individual animals there was a correlation between the fractional decrease in performance after drug and levels of transgene expression in the floccular-nodular lobe of the cerebellum, which contains the circuits involved in balance.

Together, these results show that motor learning and motor performance on the Rotarod task depend acutely on Purkinje cell output. These experiments further demonstrate that VAMP/Syb MIST specifically perturb the function of neural circuits in vivo with temporal precision on the order of a few hours. These data show that the methods of the invention can be used in vivo on rapidly inducible and reversibly time scales.

Experimental Procedures

For cerebellar targeting, VAMP-FKBP(36V)₂-HA-IRES2-EGFP-WPRE was subcloned into the pL7-DATG/1B vector kindly provided by Dr. J. Oberdick (Zhang et al., Histochem. Cell. Biol. 115: 455-64 (2001)). The transgene was separated from the vector by digestion at flanking sites, gel purified, and microinjected into the pronuclei of fertilized eggs. All of the transgenic mice used in this study were maintained in strict accordance with NIH and institutional animal care guidelines.

Rotarod testing was performed as described (Rustay et al., Behav Brain Res 141:237-49 (2003)). AccuRotor RotaRod (Accuscan Instruments, Columbus, Ohio) with 6.3 cm diameter dowels was used for the experiments. Dowel surfaces were covered with 320 grit sandpaper to provide a uniform surface. Animals were extensively pre-handled to reduce the stress and acquainted with the task. Surgery was performed to implant a cannula (Plastics One, Roanoke, Va.) into the scull for injections into the lateral ventricle. The animals were allowed to recover from surgery, and re-adjust to the task.

For the experiment during learning, transgenic animals or wild-type littermates were placed on the rod rotating at a constant speed of 30 rpm. Performance was scored from the very first session. One session of 10 runs was performed daily with five best runs being scored for each animal. After the first session, animals were injected into the lateral ventricle at 1 mm lateral, 0.2 mm caudal of Bregma with 0.5 nmoles of AP20187 in a total volume of 0.25 μl using a Hamilton syringe. For the experiment following saturation of learning, animals were challenged to a ramp protocol, with the rod accelerating to 80 rpm over 1 minute.

Animals were considered to have reached saturation when the performance of all animals was not varying by more than 20% from session to session for 3 days. At that time, baseline performance was scored and animals were injected intra-ventricularly with 0.5 mmoles of AP20187 in the total volume of 1 μl over 3 minutes using a peristaltic pump. Injections were performed at the end of baseline run, about 16 hrs before the next session to reduce the effect of handling on rotarod performance.

In both experiments, latency to fall was scored, and the results were normalized for each animal to the mean score of the session on the day prior to dimerizer injection. All data was acquired blind to the genotype of the animals. Significance of the difference between pre- and post-dimerizer scores as well as between transgenic and wild type littermates was analyzed using paired t-test. For all experiments, Significance was set at p<0.05 and evaluated using student t-tests. Error bars denote standard error of the mean.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of inhibiting synaptic transmission of a neuron, comprising:
providing a neuron comprising a polynucleotide encoding a fusion protein comprising (i) a ligand-binding domain that binds to a selected ligand, and (ii) a synaptic protein domain, wherein the selected ligand binds to and oligomerizes the fusion protein; and
administering to the neuron the selected ligand to oligomerize the fusion protein, thereby inhibiting synaptic transmission of the neuron.

2. The method of claim 1, wherein the synaptic protein domain comprises Synaptobrevin/VAMP2, SNAP-25, Syntaxin, Synaptophysin, or RIM1.

3. The method of claim 1, wherein the ligand-binding domain comprises FKBP or a variant thereof.

4. The method of claim 1, wherein the neuron is in a mammal.

5. A method of inhibiting synaptic transmission of a neuron, comprising:
providing a neuron comprising
(1) a first polynucleotide encoding a first fusion protein comprising (i) a first ligand-binding domain that binds to a selected ligand, and (ii) a synaptic protein domain, and
(2) a second polynucleotide encoding a second fusion protein comprising (i) a second ligand-binding domain that binds to the selected ligand, and (ii) a mislocalizer domain,
wherein the selected ligand binds to and crosslinks the first and second fusion proteins; and
administering to the neuron the selected ligand to crosslink the first and second fusion proteins, thereby inhibiting synaptic transmission of the neuron.

6. The method of claim 5, wherein the synaptic protein domain comprises a synaptic vesicle protein.

7. The method of claim 6, wherein the synaptic vesicle protein is Synaptophysin or Synaptobrevin/VAMP2.

8. The method of claim 5, wherein the mislocalizer domain comprises the transmembrane domain of a cell surface protein.

9. The method of claim 8, wherein the cell surface protein is Syntaxin.

10. The method of claim 5, wherein the mislocalizer domain comprises a cytoskeletal matrix protein.

11. The method of claim 10, wherein the cytoskeletal matrix protein is RIM1.

12. The method of claim 5, wherein the first and second ligand-binding domains comprise FKBP and FRB, or variants thereof.

13. The method of claim 5, wherein the neuron is in a mammal.

14. A method of identifying a synaptic protein whose oligomerization inhibits synaptic transmission of a neuron, the method comprising:
providing a neuron comprising a polynucleotide encoding a fusion protein comprising (i) a ligand-binding domain that binds to a selected ligand, and (ii) a domain comprising a candidate synaptic protein, wherein the selected ligand binds to and oligomerizes the fusion protein;
administering to the neuron the selected ligand to oligomerize the fusion protein; and
detecting synaptic transmission of the neuron, wherein reduction in the transmission indicates that the candidate synaptic protein inhibits synaptic transmission of the neuron when oligomerized.

15. The method of claim 14, wherein the ligand-binding domain comprises FKBP or a variant thereof.

16. A method of identifying a synaptic protein whose crosslinking to another cellular protein inhibits synaptic transmission of a neuron, the method comprising:
providing a neuron comprising
(1) a first polynucleotide encoding a first fusion protein comprising (i) a first ligand-binding domain that binds to a selected ligand, and (ii) a domain comprising a candidate synaptic protein, and
(2) a second polynucleotide encoding a second fusion protein comprising (i) a second ligand-binding domain that binds to the selected ligand, and (ii) a domain comprising the cellular protein,
wherein the selected ligand binds to and crosslinks the first and second fusion proteins;
administering to the neuron the selected ligand to crosslink the first and second fusion proteins; and
detecting synaptic transmission of the neuron, wherein reduction in the transmission indicates that the candidate synaptic protein inhibits synaptic transmission of the neuron when crosslinked to the cellular protein.

17. The method of claim 16, wherein the first and second ligand-binding domains are FKBP and FRB, or variants thereof.
